(19) United States
(12) Patent Application Publication
(10) Pub. No.: US 2012/0157754 A1
(43) Pub. Date: Jun. 21, 2012

(54) COMPACT BATTERY AND CONTROLLER MODULE FOR A TRANSCUTANEOUS ENERGY TRANSFER SYSTEM

(75) Inventor: Ralph L. D'Ambrosio, Wenham, MA (US)

(73) Assignee: ABIOMED, INC., Danvers, MA (US)

(21) Appl. No.: 13/328,598

(22) Filed: Dec. 16, 2011

Related U.S. Application Data
(60) Provisional application No. 61/425,160, filed on Dec. 20, 2010.

(51) Int. Cl. A61M 1/12

(52) U.S. Cl. 600/16

(57) ABSTRACT

A compact implantable controller and battery module for a transcutaneous energy transfer (TET) system is provided having a single biocompatible housing encasing an energy storage device, a power control module, and a device control module. The power control module controls energy transfer to the storage device during charging and monitors power consumption of a cardiac assist device. The device control module controls and monitors the operation of a cardiac assist device.

### RELATED APPLICATIONS

This application claims priority to U.S. Provisional Application Ser. No. 61/425,160, filed on Dec. 20, 2010, and entitled “A Compact Battery and Controller Module for a Transcutaneous Energy Transfer System.”

### FIELD

The invention relates to transcutaneous energy transfer (TET) devices and more particularly to an improved integrated battery and controller module contained in a single biocompatible housing.

### BACKGROUND

Many medical devices adapted for implantation also have high power requirements and must be frequently connected to external power sources. Inductively coupled transcutaneous energy transfer (TET) systems are increasingly popular for use in connection with these high-power implantable devices. A TET system may be employed to supplement, replace, or charge an implanted power source, such as a rechargeable battery. Unlike other types of power transfer systems, TET systems have an advantage of being able to provide power to the implanted electrical and/or mechanical device, or recharge the internal power source, without puncturing the skin. Thus, possibilities of infection are reduced and comfort and convenience are increased.

TET devices include an external primary coil and an implanted secondary coil, separated by intervening layers of tissue. The primary coil is designed to induce alternating current in the subcutaneous secondary coil, typically for transformation to direct current to power an implanted device. TET devices therefore also typically include an oscillator and other electrical circuits for providing appropriate alternating current to the primary coil. These circuits typically receive their power from an external power source.

Prior art TET systems also include several additional components implanted within a patient's body. These include a controller, configured to drive and monitor a blood pump or other implantable device, a rechargeable battery, an internal TET coil, and a blood pump. In prior art TET systems, the controller and rechargeable battery pack are separate units installed in different locations within a patient's body. This configuration is disadvantageous because it requires additional surgery to implant the multiple devices, additional space within a patient's body to accommodate the modules and their housings, and additional cabling and connectors running through a patient's body to connect the multiple devices. All of these factors increase the risk of complications for the patient.

### SUMMARY

To overcome the above and other drawbacks of conventional systems, the present invention provides a compact high-energy battery and controller module for use in a transcutaneous energy transfer system that places all of the controller circuitry along with the rechargeable battery pack inside a single housing adapted for disposition inside a patient's body.

One aspect of the invention provides an implantable controller for controlling a cardiac assist device including a single biocompatible housing encasing an energy storage device, a power control module, and a device control module. The power control module controls energy transfer to the storage device during charging and monitors power consumption during use of the cardiac assist device. The device control module controls and monitors the operation of the cardiac assist device.

In one embodiment, the controller also includes a communications module for transmitting information to external diagnostic or control equipment.

In another embodiment, the controller includes a microprocessor for controlling the power control module and device control module.

In still another embodiment, the controller includes an interface for connecting one or more coils adapted for disposition in a patient and configured to produce electric current in the presence of a time-varying magnetic field. In some embodiments, the interface can include a glass-to-metal hermetic connector for connecting to one or more secondary coils, or other implanted components.

In some embodiments, the single biocompatible housing can be formed from any of titanium, stainless steel, epoxy, plastic, ceramic, glass, or polyurethane. In certain embodiments, the housing can include large-radius corners and edges configured to prevent tissue necrosis when implanted in a patient.

### BRIEF DESCRIPTION OF THE DRAWINGS

The invention will be more fully understood from the following detailed description taken in conjunction with the accompanying drawings, in which:

FIG. 1 is a diagram of a TET system including a controller of the present invention;

FIG. 2 is an illustration of an exemplary implantable secondary coil for use in a TET system;

FIG. 3 is an illustration of an exemplary primary coil for use in a TET system;

FIG. 4 is a front perspective view of an exemplary ventricular assist device powered by a TET system; and

FIG. 5 is a diagram of an exemplary implantable controller containing power and control circuitry, as well as a rechargeable battery pack.

### DETAILED DESCRIPTION

Certain exemplary embodiments will now be described to provide an overall understanding of the principles of the devices disclosed herein. One or more examples of these embodiments are illustrated in the accompanying drawings. Those skilled in the art will understand that the devices specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

A transcutaneous energy transfer (TET) system works by inductively coupling a primary coil to a secondary coil. The primary coil, configured for disposition outside a patient, is connected to a power source and creates a time-varying magnetic field. When properly aligned with a secondary coil, the time-varying magnetic field from the primary coil induces an alternating electric current in the secondary coil. The secondary coil(s) is configured for implantation inside a patient and can be connected to a controller that harnesses the electric current and uses it to, for example, charge a battery pack or power an implantable device like a ventricular assist device (VAD), or other medical assist device. By utilizing induction to transfer energy, TET systems avoid having to maintain an open passage through a patient's skin to power an implantable device.

Prior art TET systems feature a separate implantable controller and rechargeable battery pack due to size (only certain volumes can be implanted in one location of the body) and efficiency constraints (surface temperature limitations can result in multiple containers). These separate units are implanted in different locations in a patient's body. A disadvantage of these prior art TET systems is that multiple implantation sites must be prepared and surgically accessed to install the TET system. In addition, the multiple components occupy a large amount of space within a patient's body and require complex cabling be run through the body to connect the various devices. This increases the risk of complications or discomfort for patients receiving the implanted TET system.

The present invention solves these problems and reduces risks to a patient by providing a compact high-energy battery and controller module in a single biocompatible housing. Such a configuration allows for the use of a single implantation site for the battery and all controller circuitry. In addition, the integration of all components within a single housing eliminates the need for cabling because all connections between the components are located inside the housing as well.

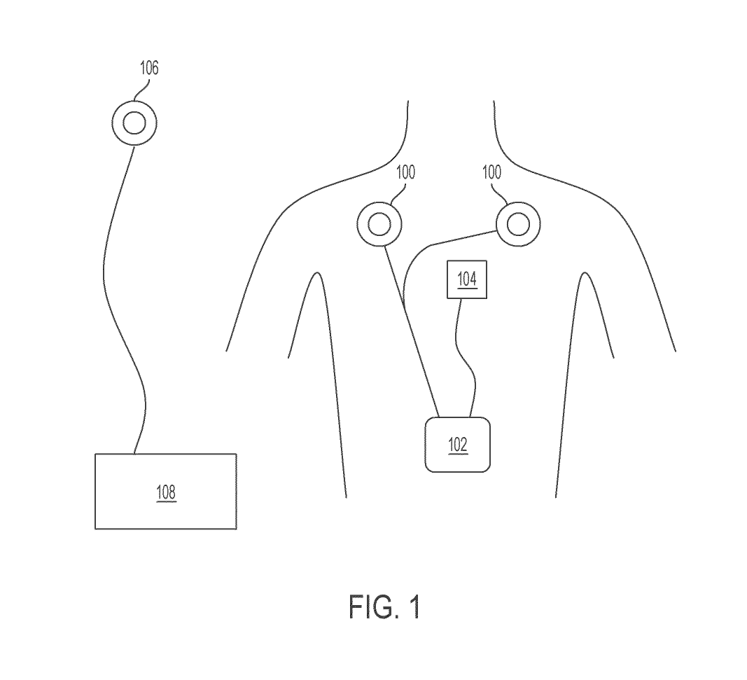

FIG. 1 shows a diagram of an exemplary TET system using the controller of the present invention. An implantable device comprises a single or plurality of secondary coils 100 adapted for disposition in a patient. The secondary coil(s) are connected to a controller and battery module 102 of the present invention that is adapted to receive electric current from the single or plurality of secondary coils for use or storage. The controller can then direct the electric current to, for example, charge the integrated battery or power a ventricular assist device 104 or other implantable device.

FIG. 1 also shows an exemplary embodiment of primary coil 106 that is adapted to remain outside the body and transfer energy inductively to the secondary coils. Primary coil 106 is connected to an external power source, which can include, for example, conditioning and control circuitry. Optionally, more than one primary coil 106 can be used simultaneously with the multiple secondary coils 100 to reduce the time required to charge an implanted battery.

In use, primary coil(s) 106 are placed over the area of secondary coil(s) 100 such that they are substantially in axial alignment. Power source 108, which can include conditioning circuitry to produce a desired output voltage and current profile, is then activated to produce a time-varying magnetic field in the primary coil(s) 106. The time-varying magnetic field induces an electric current to flow in the secondary coils 100 and the current is subsequently distributed to controller and battery module 102 and any attached ventricular assist devices 104.

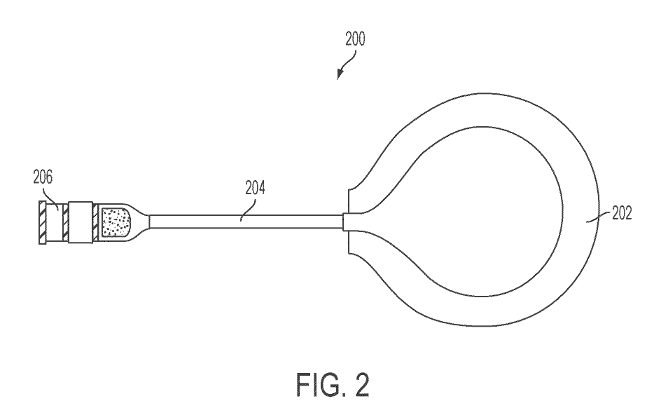

FIG. 2 illustrates an exemplary secondary coil 200 adapted for disposition in a patient. Secondary coil 200 features a coil portion 202 consisting of several turns of conductive wire, a connecting portion 204, and an optional interface portion 206. Coil portion 202 can vary in size and turns of wire depending on numerous factors such as the intended implantation site. In an exemplary embodiment, coil portion 202 comprises 12 turns of Litz wire in a two-inch diameter coil. In addition to the wire, the coil 202 can contain a ferrous core and electronic circuitry which rectifies the AC current and communicates with the external coil and driver to provide a regulated DC output voltage. An exemplary secondary coil is described in U.S. Patent Pub. No. 2003/0171792, which is incorporated herein by reference.

The coil portion 202 is electrically coupled to the connecting portion 204, which can be formed from a segment of the same wire used to form the coil portion. The length of connecting portion 204 can also vary based on, for example, the distance from the implantation site of a secondary coil to that of a controller.

Connecting portion 204 is also electrically coupled to optional interface portion 206. Interface portion 206 is used to connect the secondary coil 200 to a controller and battery module 102. The interface portion can include any electrical connector known in the art to facilitate modular connection to a controller and battery module 102, or can consist of a terminal end of the connecting portion 204 that is capable of being electrically connected to a controller.

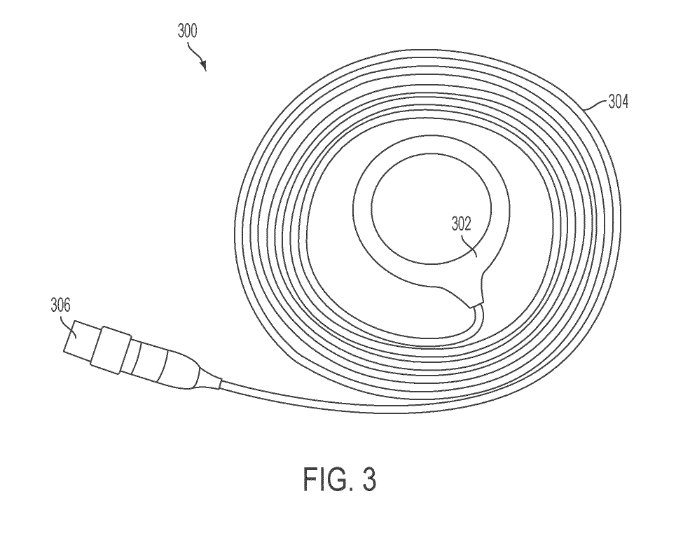

FIG. 3 shows an exemplary primary coil 300 configured to transmit transcutaneous energy to a secondary coil like that illustrated in FIG. 2. Similar to secondary coil 200 in FIG. 2, primary coil 300 can include a coil portion 302, a connecting portion 304, and an interface portion 306. Primary coil 300 is adapted for disposition outside the patient, however, and induces electric current in secondary coil 200 by emitting a time-varying magnetic field from coil portion 302.

Coil portion 302 can vary in size and turns of wire depending on several factors including, for example, the size of any secondary coils it will be used with. Coil portion 302 is electrically coupled to connecting portion 304. Connecting portion 304 can be formed from a portion of the wire used to form coil portion 302. Connecting portion 304 can vary in length depending on any of several factors including, for example, how far a patient is from a power source. Connecting portion 304 is in turn electrically coupled to interface portion 306, which is adapted to connect to a power source (or associated conditioning or control circuitry) like power source 108 of FIG. 1. Interface portion 306 can include any electrical connector known in the art to facilitate modular connections to external power source 108, or can consist of a terminal end of connecting portion 304 that is adapted to be electrically connected to power source 108.

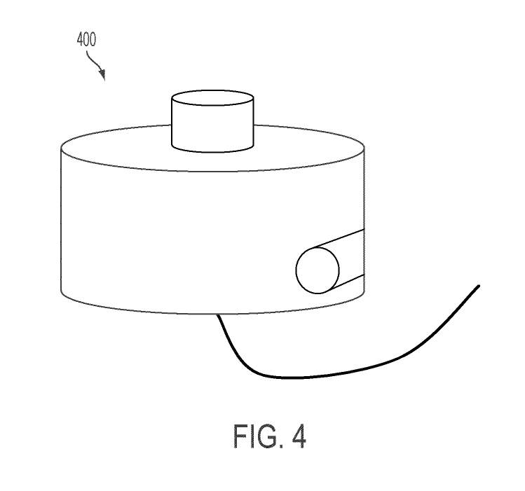

Primary coil 300 is used to transfer power transcutaneously in order to ultimately support an implantable device like the ventricular assist device (VAD) 400 depicted in FIG. 4. The ventricular assist device 400 aids the heart in circulating blood through the body. The integration of sufficient battery capacity within the body and a power-efficient assist device (e.g., 5-6 Watt electrical input) can allow a patient to be mobile without any external power source or primary coil attachment for long periods of time. This results in an unsurpassed quality of life for patients using these systems.

While a ventricular assist device is an exemplary embodiment of an implantable device that can benefit from TET systems, it is by no means the only implantable device that can be powered in this way. Other cardiac assist devices, as well as many other types of powered implantable devices, can be used with the controller of the present invention. Exemplary embodiments of the controller and battery module of the present invention contain modular circuit components so that control circuitry for various types of implantable medical devices can easily be incorporated into the housing.

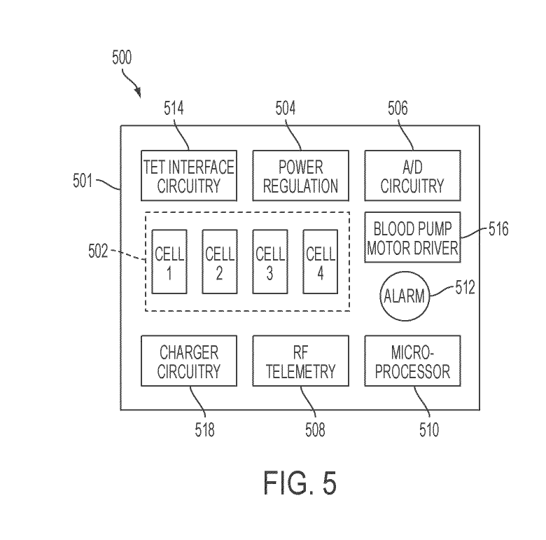

FIG. 1 shows the secondary coils 100 connected to the ventricular assist device 104 via a controller and battery module like that illustrated in FIG. 5. FIG. 5 depicts an integrated controller and battery module 500 that is adapted for disposition in a patient. The controller and battery module includes a biocompatible housing 501 that encapsulates the rechargeable battery cells 502 and all of the controller circuitry 504-518.

The controller and battery module's housing is designed for biocompatibility. Exemplary materials that can be used in the housing include one or more of titanium, stainless steel, epoxy, plastic, ceramic, glass, or polyurethane. By way of example, a housing can be formed from titanium with a glass-to-metal hermetic connector to interface with other implanted components. The controller and battery module housing can be any size suitable for implantation in a patient. An exemplary housing can measure about 3.75×about 3.275×about 1.25 inches. Exemplary embodiments of the controller and battery module housing can also have additional bio-compatibility features such as large-radius rounded corners and edges to prevent internal damage due to tissue necrosis or poor tissue in-growth.

The controller and battery module 500 can include rechargeable battery cells 502, a power control module that comprises TET interface circuitry 514, power regulation circuitry 504, and charger circuitry 518, as well as a device control module that comprises A/D circuitry 506 and blood pump motor driver 516. The controller and battery module 500 can also include a microprocessor 510, RF telemetry module 508, and alarm module 512.

The controller and battery module 102 can include an interface for connecting to a plurality of secondary coils 100 and receiving electric current therefrom. The rechargeable battery cells 502 can be charged using the electric current received from the secondary coil(s) 100. Electric current received from the secondary coil(s) 100 is processed through the TET interface circuitry 514 and further conditioned for use with the battery cells 502 through the charger circuitry 518 or to power the internal electronics and ventricular assist device 104 by power regulation circuitry 504. Power regulation circuitry 504 can contain any of several circuit designs known in the art that are effective to convert the voltage and current received from the TET interface circuitry 514 into a desired output voltage and current that can be used to power the internal electronic circuitry 506, 508, 510, 512 and the ventricular assist device 104 via the blood pump motor driver 516.

Controller 500 can also include a device control module comprising A/D circuitry 506 and blood pump motor driver 516 that is configured to control the ventricular assist device 104. The device control module can include monitoring features so that any failures in the ventricular assist device 104 can be detected in the controller 500. The controller 500 can further include a microprocessor 510 that coordinates functions executed by the charger circuitry 518, power regulation circuitry 504, blood pump motor driver circuitry 516, and A/D circuitry 506.

The processor 510 also monitors the function of secondary coils 100 and ventricular assist device 104. If a fault is detected in either component, processor 510 can utilize RF telemetry module 508 to allow it to communicate fault information with a user via an external display or control console. The display or control console could take the form of a common desktop computer, mobile phone, PDA, bed-side control console, or any other type of computing or signaling device known in the art. The fault information communicated to a user can also be in the form of an alarm sounded by a display or control console as described above. Alternatively, controller 500 can include an alarm module 512 that can sound an auditory or vibratory alarm in the event of a failure. In addition, the external power source 108 can also be configured to detect a fault in a coupled secondary coil 100 and alert a patient accordingly.

The controller of the present invention provides several benefits over prior art TET systems. For example, the controller of the present invention reduces the amount of space required in a patient's body as well as the amount of surgery required to implant a TET system. In addition, the controller eliminates the need for complex cabling running through a patient's body between the controller and battery pack. Components within the controller can be positioned such that the largest heat source is positioned against blood-rich tissue when implanted in order to avoid hot spots. Additionally, controller components can be integrated such that even temperature distribution is obtained to allow for maximum heat transfer into the adjacent tissue without localized hot spots. Finally, the controller of the present invention provides a modular base that can be configured to control a variety of implantable medical devices. All of these features reduce the risk of complications for patients receiving implantable TET systems.

All papers and publications cited herein are hereby incorporated by reference in their entirety. One skilled in the art will appreciate further features and advantages of the invention based on the above-described embodiments. Accordingly, the invention is not to be limited by what has been particularly shown and described, except as indicated by the appended claims.

What is claimed is:

1. An implantable controller for controlling a cardiac assist device, comprising:
a single biocompatible housing encasing:
an energy storage device (a rechargeable battery pack);
a power control module for controlling energy transfer to the storage device during charging and for monitoring power consumption during use of the cardiac assist device; and
a device control module for controlling and monitoring the operation of a cardiac assist device.

2. The controller of claim 1, wherein the controller further comprises a communications module for transmitting information to external diagnostic or control equipment.

3. The controller of claim 1, wherein the controller further comprises a microprocessor for controlling the power control module and device control module.

4. The controller of claim 1, wherein the controller further comprises an interface for connecting one or more coils adapted for disposition in a patient and configured to produce electric current in the presence of a time-varying magnetic field.

5. The controller of claim 4, wherein the interface comprises a glass-to-metal hermetic connector for connecting to the one or more coils.

6. The controller of claim 1, wherein the housing is formed from any of titanium, stainless steel, epoxy, plastic, ceramic, glass, or polyurethane.

7. The controller of claim 1, wherein the housing includes large-radius corners and edges configured to prevent tissue necrosis when implanted in a patient.
